(19) United States
(12) Patent Application Publication
(10) Pub. No.: US 2006/0142703 A1
(43) Pub. Date: Jun. 29, 2006

(54) Catheter aperture with related structures and method

(75) Inventors: Matthew P. Carter, Dobson, NC (US); Kenneth C. Kennedy II, Clemmons, NC (US); Frederick B. Haller, Winston-Salem, NC (US); David M. Hardin JR., Winston-Salem, NC (US); Cheri L. Matney, Walnut Cove, NC (US); Brian K. Rucker, King, NC (US); David F. Waller, Winston-Salem, NC (US); Jacques Deviere, Bruxelles (BE); Patricia J. Chilton, Mt. Airy, NC (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/294,947

(22) Filed: Dec. 6, 2005

Related U.S. Application Data
(60) Provisional application No. 60/633,793, filed on Dec. 7, 2004.

(51) Int. Cl. A61M 25/00

(52) U.S. Cl. 604/264; 604/103.04; 264/138

(57) ABSTRACT

An elongate catheter shaft having a side port aperture through a side wall of the catheter shaft between proximal and distal ends of the shaft. The side port aperture is open to a lumen. Embodiments of the present invention are directed to stiffening structure disposed in the immediate vicinity of the side port aperture. The stiffening structures may be disposed on or be continuous with, for example, an exterior surface, an interior lumenal surface, within a wall of the catheter shaft, or some combination thereof. The stiffening structures described herein are directed to biasing the catheter shaft in the region of a side port aperture in a straight or curved configuration that resists undesired flexure in the region of the side port aperture.

### CROSS-REFERENCE TO RELATED APPLICATIONS

This application claims priority to U.S. Provisional Application Ser. No. 60/633,793, filed Dec. 7, 2004.

### BACKGROUND

The present application relates to medical catheters. The present application relates more specifically to medical catheters having a wire guide lumen and a side port aperture that is useful for introduction of a wire guide into the lumen in a configuration commonly known as “rapid exchange,” “short wire guide,” or “monorail”, and that is also useful for other applications in minimally invasive surgical procedures. In particular the present application relates to methods and structures for forming a side port aperture in a catheter shaft and reinforcing the catheter shaft in the region of the side port aperture.

Medical delivery catheters are well known in the art of minimally invasive surgery for introduction of fluids and devices to sites inside a patient's body. A well-established technique, known as “long wire guide,” for guiding a delivery catheter to a target site in a patient body includes: (1) positioning a wire guide along a desired path to the target site; (2) retaining a proximal portion of the wire guide outside the body; (3) threading the delivery catheter, which has a wire guide lumen throughout its length, onto the proximal end of the wire guide; and (4) advancing the catheter along the wire guide to the treatment site.

One example of a desired path to a target site is the passage through a working lumen or channel of an endoscope to a biliary duct in a gastroenterological application. Another example of a desired path is through an endovascular lumen to an occluded coronary artery in a cardiological application. The delivery catheter may have a treatment device such as a stent or fluid-inflatable balloon disposed at its distal end for deployment at a target site (e.g., an occluded biliary duct or coronary artery). The catheter may also have a tool such as a cutting wire or cutting needle disposed at or near its distal end (e.g., a papillotome, sphincterotome, etc.), or the catheter may have an aperture for the delivery of a fluid through a second lumen (e.g., radio-opaque fluid for contrast fluoroscopy, adhesive or gelling agent for delivery to a target site, etc.).

Procedures that employ wire guides often require exchange of treatment appliances. For example, a balloon catheter may be replaced with a stent deployment catheter. In a typical application of such a procedure, a balloon catheter is directed to the site of a stenosis (e.g. in an artery, biliary duct, or other body lumen) as described above. Fluid is then used to inflate the balloon so as to dilate the stenosis. Some procedures are effectively concluded at this point. However, many procedures follow dilation of the stenotic stricture with the placement of a stent to maintain patency of the re-opened lumen. This requires that the balloon catheter be withdrawn to allow introduction of a stent-deployment catheter. It is preferable that the wire guide remain in place for guidance of the stent-deployment catheter without having to re-navigate the wire guide back into to the newly re-opened lumen. In order to prevent undesired displacement of the wire guide, any exchange of long wire guide catheters requires that the proximal portion of the wire guide extending out of the patient's body (or endoscope, depending on the entry point for the desired path to the target site) must be longer than the catheter being “exchanged out” so that control of the wire guide may be maintained as the catheter is being removed. Likewise, the wire guide must be grasped while the entire catheter being “exchanged in” is threaded onto it and directed along the desired path to the target site. In other words, for the operating physician and assistant to be able to hold the wire guide in place while removing one catheter for replacement with another, each of the catheters must be shorter than the portion of the wire guide that is exposed outside the patient's body (and, if used, outside the endoscope). Put another way, the wire guide must be about twice as long as a catheter that is being used over that wire guide. Additionally, in the case of gastrointestinal endoscopy, even more wire guide length is necessary. This is because the shaft of the endoscope through which the wire guide and catheters are placed must have a length outside the body for manipulation and control, and the catheter itself must have some additional length outside of the endoscope for the same reason. As those skilled in the art will appreciate, wire guides having the necessary “exchange length” are cumbersome and difficult to prevent from becoming contaminated.

An alternative technique for guiding a delivery catheter to a target site in a patient body utilizes catheters having a relatively short wire guide lumen in catheter systems commonly referred to as “rapid exchange,” “short wire guide,” or “monorail” systems. In such systems, the wire guide lumen extends only from a first lumen opening spaced a short distance from the distal end of the catheter to a second lumen opening at or near the distal end of the catheter. As a result, the only lumenal contact between the catheter's wire guide lumen and the wire guide itself is the relatively short distance between the first and second lumen openings. Several known advantages are conferred by this configuration. For example, the portion of the wire guide outside the patient's body may be significantly shorter than that needed for the “long wire configuration.” This is because only the wire guide lumen portion of the catheter is threaded onto the wire guide before directing the catheter through the desired path (e.g., a working lumen of an endoscope, an endolumenal passage, etc.) to the target site. By way of illustration, the prior art pictured in FIGS. 1A and 1B illustrate the distal ends of two different types of typical catheters. FIG. 1A shows the distal end of a prior art long-wire catheter shaft 100 with a wire guide 102 disposed in a lumen 104. The lumen 104 extends substantially to the proximal end of the catheter shaft 100 (not shown). FIG. 1B shows the distal end of a prior art short-wire catheter shaft 110 with a side port aperture 111 and a wire guide 112 disposed in a lumen 114. The length of the lumen 114, and consequently the exchange length of the catheter 110, is substantially shorter than that of the catheter 100 shown in FIG. 1A. In addition to a shorter exchange length, the catheter 110 (FIG. 1B) has a reduced surface contact between the wire guide and catheter lumen that results in a reduced friction between the two. This can result in an eased threading and exchange process by reducing the time and space needed for catheter exchange. This economy of time and space is advantageous for minimally invasive surgeries by reducing the likelihood of contamination and reducing the total time and stress of completing surgical procedures. On occasion, when advantageous, the catheter may be left in place, and the first wire guide removed and replaced with a second wire guide or the wire guide lumen may be used for another purpose such as injecting a contrast media.

In certain rapid exchange catheter configurations, the wire guide lumen is open to a side port aperture in the side of the catheter between its proximal and distal ends. In one such configuration, the wire guide lumen only extends from the side port aperture to an opening at the distal end. An example of this type of rapid exchange catheter is illustrated in FIG. 1B.

In another type of rapid exchange catheter configuration, the wire guide lumen extends through the length of the catheter from near its proximal end to its distal end. A side port aperture between the proximal and distal ends opens into the wire guide lumen. This side port aperture allows the catheter to be used in a short wire guide configuration, while the full-length wire guide lumen allows the catheter to be used in a long wire guide configuration. This wire guide lumen configuration is referred to as “convertible” or “dual use.” An example of this type of catheter is illustrated in FIG. 1C, which shows the distal end of a prior art “convertible” catheter shaft 120 with a wire guide 122 disposed through a side port aperture 121 and into a wire guide lumen 124. Specifically, a wire guide may run through substantially the entire length of the wire guide lumen, or the wire guide may run only through the portion of the lumen between the distal end and the side port aperture.

While offering advantages as explained above, the configurations having a side port aperture are prone to undesirable flexure (e.g., excessive bending, kinking, twisting, or binding) in the region around the aperture. This is often due to the lack of full columnar support in the region of the side port aperture. Such undesired flexure can have several negative consequences. For example, kinking or excessive flexure of the catheter may cause one or more lumens to be closed off—thereby preventing their use, or may cause a non-smooth edge to be formed adjacent the aperture that could cause damage (e.g., injure the endolumenal passage of a patient or damage the working channel of an endoscope through which the catheter shaft is being passed).

In addition, a dual use configuration catheter tends to allow a wire guide being passed from the proximal end through the length of a catheter (in place in the body) to inadvertently pass out through the side port aperture, rather than proceeding to the end of the wire guide lumen (e.g., when replacing a primary wire guide with a second, different diameter wire guide). This presents an obvious problem in that the wire guide, to be useful, must exit the wire guide lumen of the catheter via the desired aperture.

Therefore, it is an object of the present invention to provide stiffening structure for preventing undesirable flexure of the catheter shaft in the region near the side port aperture providing access into the lumen of the catheter. It is a further object of the present invention to provide structure associated with the side port aperture such that, in a dual use wire guide configuration, a wire guide being directed from the proximal end through the wire guide lumen has a reduced likelihood of exiting out through the side port aperture. It is contemplated that the aforementioned side port aperture and catheter lumen described will have applications other than for use with a wire guide.

### BRIEF SUMMARY

In one aspect, the present invention includes a catheter having an elongate shaft with proximal and distal ends, a first lumen extending through at least a portion of the shaft and defined by a wall, an aperture between the proximal and distal ends and open through the wall to the first lumen, an outer circumference, and stiffening structure disposed near the aperture. In another aspect the present invention includes a method of forming a reinforced aperture in a shaft of a catheter for promoting a desired directional passage of a wire guide in a desired path. The method includes the steps of (A) providing a catheter having a shaft comprising a first lumen defining an interior surface, a proximal end, a distal end, an outer circumference, and an exterior surface; (B) cutting the exterior surface near the distal end to form an aperture open from the exterior surface to the first lumen; and (C) providing a reinforcing band immediately adjacent the aperture.

### BRIEF DESCRIPTION OF THE DRAWINGS

FIG. 1A illustrates the distal portion of a typical prior art long-wire catheter shaft;

FIG. 1B illustrates the distal portion of a typical prior art short-wire catheter shaft;

FIG. 1C illustrates the distal portion of a typical prior art convertible catheter shaft;

FIGS. 2A-2C illustrate embodiments of a catheter shaft having stiffening structure comprising chemical compositions;

FIGS. 3A-3D show embodiments of a catheter shaft having stylet stiffening structure;

FIGS. 4A-4E illustrate embodiments of a catheter shaft having stiffening structure on, in, or around a lumenal surface;

FIGS. 5A-5C illustrate embodiments of a catheter shaft having stiffening structure disposed in a lumen or a septum;

FIGS. 6A-6F show embodiments of a catheter shaft having a side port aperture and relate to stiffening structure for support around the aperture;

FIG. 6G shows an embodiment of a catheter shaft having a side port aperture, but lacking means to prevent misdirection of a wire guide;

FIG. 6H shows an embodiment of a catheter shaft having a side port aperture and a stiffening structure for support around the aperture; and

FIGS. 7A-7C illustrate a method for creating a side port aperture and placing a support band onto a catheter shaft.

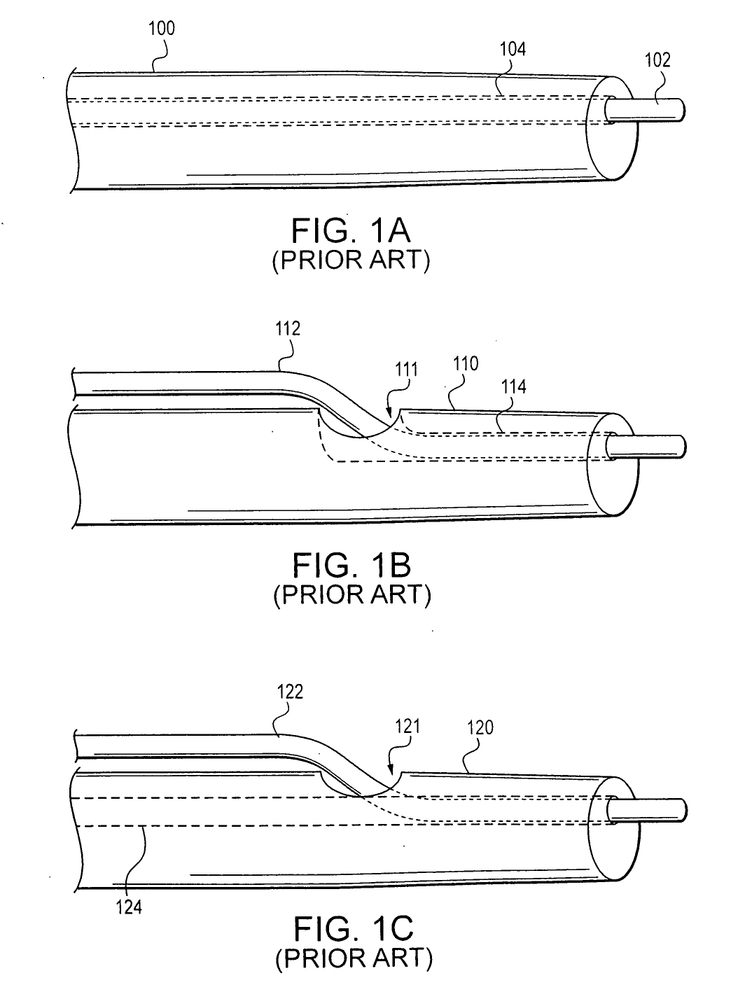

### DETAILED DESCRIPTION OF EXEMPLARY EMBODIMENTS OF THE PRESENT INVENTION

The embodiments of the present invention disclosed herein are generally described in connection with an elongate catheter shaft having a side port aperture through a side wall of the catheter shaft and open to a lumen within the catheter shaft. The side port aperture is typically located between the proximal and distal ends of the shaft. The embodiments of the present invention provide stiffening structure disposed in the immediate vicinity of the side port aperture. More specifically, the embodiments disclosed include stiffening structure that is immediately adjacent the side port aperture and/or that traverses the catheter shaft adjacent to or opposite of the side port aperture. As detailed herein, the stiffening structures may be disposed on or be continuous with, for example, an exterior surface of the catheter shaft, an interior lumenal surface of the catheter shaft, within a wall of the catheter shaft, or some combination thereof. The stiffening structures described herein are directed to biasing the catheter shaft in the region of a side port aperture in a straight or moderately curved configuration that resists undesired flexure.

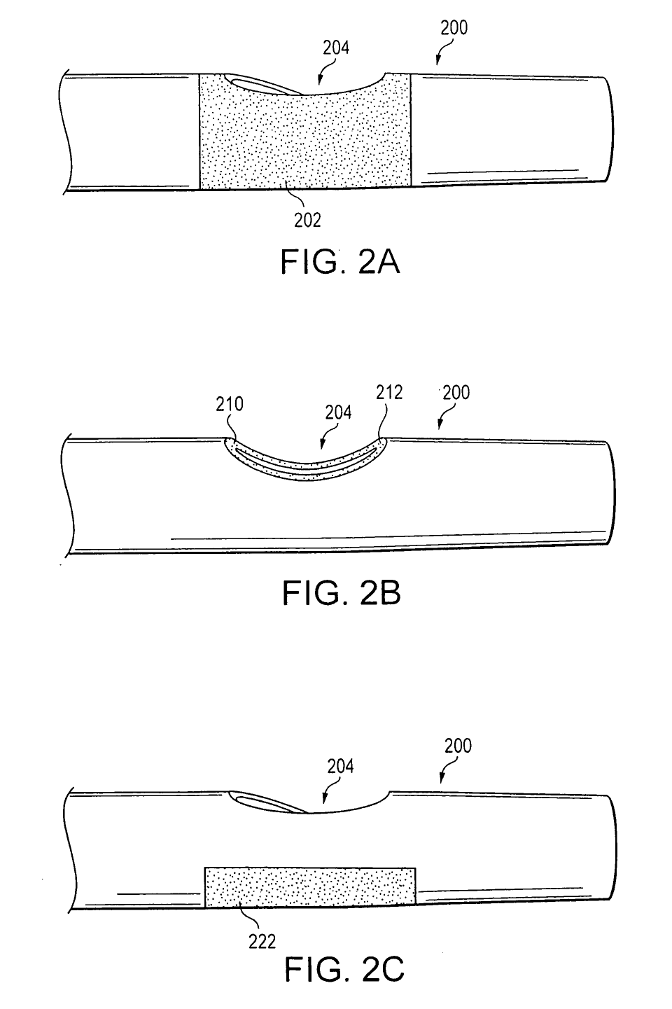

FIG. 2A shows an embodiment of a catheter shaft 200 having a stiffening structure comprising a material that is disposed on a surface of the shaft by, for example, painting, molding, or some other method of deposition. In the particular embodiment illustrated, the stiffening structure is an ink composition 202 containing particulate metal flakes to enhance its stiffening properties. The ink composition 202 is disposed about an approximately cylindrical exterior portion of the outer circumference of the catheter shaft 200 surrounding side port aperture 204 and increases the stiffness of the catheter shaft 200. FIG. 2B shows an alternative embodiment of the catheter shaft 200 wherein the stiffening structure is a layer of adhesive 210 and is disposed on or along an edge forming a lip 212 of the side port aperture 204. The stiffened lip 212 resists deformation or collapse of the aperture 204. FIG. 2C shows a different alternative of the catheter shaft 200 wherein the stiffening structure is an application of paint 222 disposed on an exterior surface of the catheter shaft 200 opposite the side port aperture 204. In other alternative embodiments, the stiffening structure can be disposed, for example, on one or both lateral sides of the catheter shaft immediately adjacent either side of the side port aperture 204, or on some combination of the above-named locations.

In further embodiments not illustrated here, the surface on which the stiffening structure is disposed is an interior surface of the catheter shaft. There are many alternative embodiments of substances and processes that can be applied in the region of the side port aperture to confer enhanced stiffness. For example, the stiffening structure can be a polymer that is painted or otherwise applied to a surface of the catheter shaft. The polymer itself may have a stiffness that enhances catheter stiffness (e.g. a cyanoacrylate that cures to produce a stiff application). The polymer may be, for example, a self-curing polymer or a mixture (e.g. bone cement) that only begins curing upon mixture and/or application. Alternatively, or in conjunction with an inherent polymer stiffness, the polymer may act mechanically to increase the catheter stiffness by thickening a region of the catheter shaft. As another example, the stiffening structure may be a composite material such as a particulates suspended in a polymer matrix (e.g. ceramic particles suspended in a latex compound) that confers stiffness when applied to the catheter shaft.

Yet another example of a stiffening structure is an application of solution-dissolved, solvent-suspended, or carrier-suspended particulates to the catheter shaft. Evaporation or other removal of the solvent or other carrier leaves stiffness-enhancing particulates disposed on the catheter shaft in the desired region. In another example of a stiffening structure, a solvent-particulate mixture is applied to the catheter shaft. The solvent acts to soften or partially dissolve a portion of the catheter shaft surface, thereby allowing the particulates to become embedded in the catheter shaft wall. The solvent is removed with a curing process (e.g. evaporation), and the resulting composite of particulates embedded in the catheter shaft wall provides an enhanced stiffness.

In yet another example of a stiffening structure, energy (e.g., heat, visible light, infrared light, ultraviolet light, RF energy, microwaves, X-rays, ultrasound waves, and any combination thereof) is selectively applied to a region of the catheter shaft in a manner causing cross-linking or other alterations within the composition of the shaft. This alteration causes a mechanical property change, enhancing the stiffness in the region to which the energy is applied. Alternatively, a chemical agent (e.g. a crosslinking agent) is applied—alone or in combination with energy or other chemical agents—to effect a change in the catheter shaft composition. In one such alternative, at least some of the chemical agent is removed, leaving the stiffened catheter shaft. In another alternative, the chemical agent bonds with the catheter shaft to confer the enhanced stiffness. In these embodiments, the material composition of the catheter shaft may be selected to provide the desired susceptibility to stiffening by a selected energy form and/or chemical agent. Those of skill in the art will appreciate that many different energy applications and chemical agents are amenable to the above-described methods.

The stiffening effect of the composition on the shaft surface may be conferred in different ways depending upon the composition and method of application. For example, application to a catheter surface in the region of a side port aperture may enhance mechanical stiffness by increasing the thickness of the catheter wall in the immediate region of the aperture (e.g., surrounding the lip of the aperture or coating at least a portion of the walls of the catheter shaft along the lateral sides of the aperture). Alternatively, or in addition, the material applied may be stiffer than the material comprising the catheter wall, thereby resulting in a combination of materials having an enhanced stiffness. Moreover, the stiffening material or method may alter the physical and/or chemical properties of the catheter shaft itself, thereby enhancing its stiffness.

Application of the stiffening structure need not significantly affect the profile of the catheter wall. For example, the material may be applied after a portion of catheter wall, such as a thin annular slice, is removed, such that the inside and/or outside diameter of the catheter shaft where the stiffening structure is applied is not significantly altered.

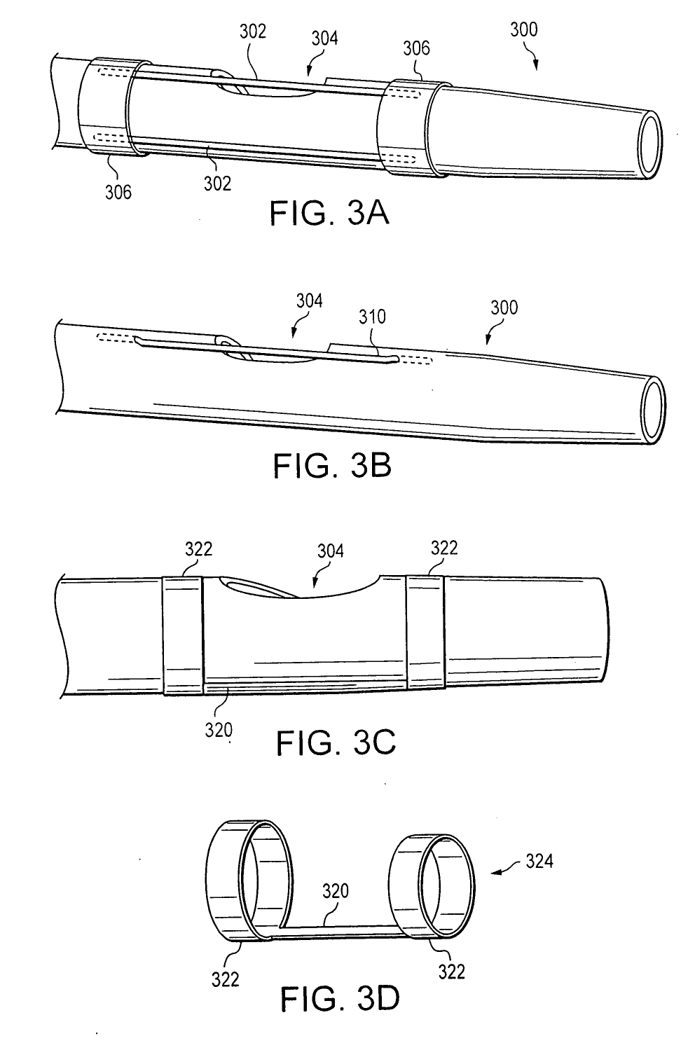

FIG. 3A shows an embodiment of a catheter shaft 300 having a stiffening structure affixed to the exterior surface of the shaft. The stiffening structure is a pair of wire stylets 302, secured at their ends by two support bands 306 to the exterior surface of catheter shaft 300. The two support bands 306 are respectively located proximally and distally of the side port aperture 304. In the illustrated embodiment, the wire stylets 302 and bands 306 are nitinol (“memory metal”). The bands 306 may be swaged, crimped, or otherwise affixed (e.g., by adhesive) to the catheter shaft 300 near the side port aperture 304.

FIG. 3B shows an alternative embodiment of the catheter shaft 300, wherein the ends of a stylet 310 are secured within the wall of the catheter shaft 300. This configuration prevents the ends of the stylet 310 from catching on, and possibly injuring, for example, the luminal wall of the patient. In alternative embodiments of the devices illustrated in FIGS. 3A-3B, more or less than two stylets may be used.

FIG. 3C shows a different embodiment wherein the stylet is a tab 320 rather than a wire. The stylet tab 320 is secured by a pair of support bands 322 to catheter shaft 300 and traverses a region opposite the side port aperture 304. In this embodiment, the bands 322 and stylet tab 320 may be formed as a one-piece stiffener 324, as shown in FIG. 3D, having a unitary construction. In such an embodiment, the one-piece stiffener 324 can be placed on the catheter shaft 300 and crimped in place. In alternative embodiments of the device illustrated in FIG. 3C, more than one stylet tab may be used.

In alternative embodiments of the catheter shaft embodiments shown in FIGS. 3A-3D, the stylets 302, 310, 320 and bands 306, 322 may be made of the same or different materials, and may comprise suitable metals such as, for example, niti (a nickel titanium alloy), or may comprise a deformable plastic material. In other alternative embodiments, the bands may be placed within the wall of the catheter shaft, or against the interior surface of the wall, so as to surround and be continuous with a radial portion of an interior lumenal surface.

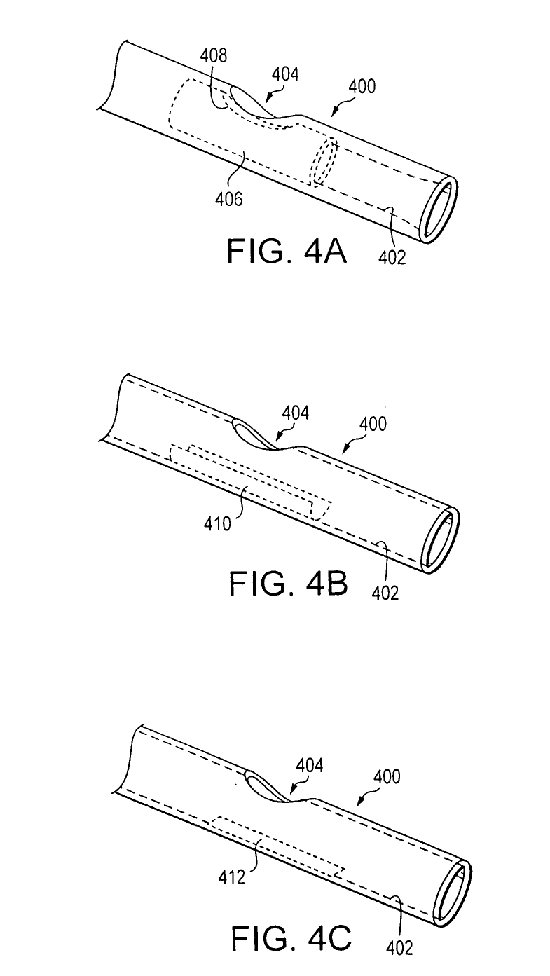

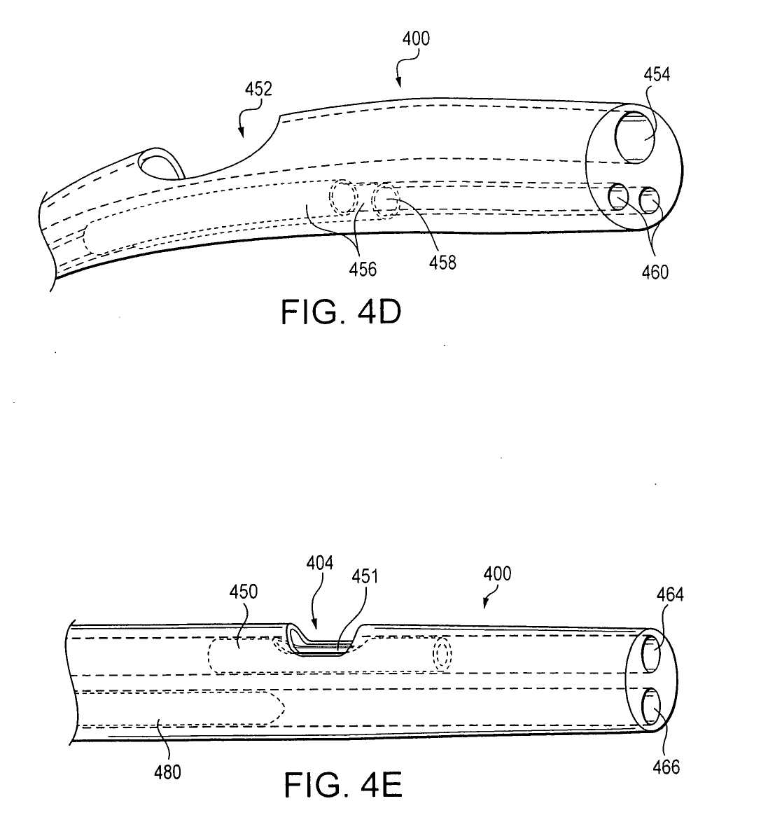

FIGS. 4A-4C illustrate embodiments of a catheter shaft 400 having a stiffening structure that is made of, for example, nitinol or another suitable stiffening material disposed on an internal surface 402 of the catheter shaft 400. More specifically, the stiffening structure comprises a lumenal surface 402 that is immediately adjacent a side port aperture 404. In the embodiment shown in FIG. 4A, the stiffening structure is a generally cylindrical cannula 406 disposed about the lumenal surface 402 of the catheter shaft 400 and having a side aperture 408 aligned with side port aperture 404 of the catheter shaft 400. The embodiment shown in FIG. 4B has a semi-cylindrical cannula stiffening member 410 disposed in the inner lumenal surface diameter 402 of the catheter shaft 400 opposite the side port aperture 404. The embodiment shown in FIG. 4C has an elongate stylet tab stiffening member 410 disposed within or adjacent to the inner surface diameter 402 of the catheter shaft 400 opposite the side port aperture 404.

In alternative embodiments shown in FIGS. 4D-4E, the stiffening structure or structures may be disposed in or about one or more lumens within a catheter shaft. FIG. 4D illustrates a catheter shaft 400 having a side port aperture 452 open to a wire guide lumen 454. Two separate cannulas, each in the form of tubular stiffeners 456 bridge the region adjacent the side port aperture 452, with inner diameters 458 of the stiffeners 456 being continuous and in fluid communication with secondary lumens 460. FIG. 4E illustrates a catheter shaft 400 having stiffening structure that is a generally cylindrical cannula 450 disposed about the inner diameter of a wire guide lumen 464. The cannula 450 has a side aperture 451 aligned with side port aperture 404 of the catheter shaft 400. The catheter shaft 400 also has another lumen 466 separate from the wire guide lumen 464. FIG. 4E also illustrates placement of a wire guide 480 in the lumen 466. This wire guide 480 may, in alternative embodiments, be some other stiffening member (e.g. a flexible metal stylet) placed in the lumen 466 (or, optionally, in the wire guide lumen 464) proximally of the side aperture 451 to enhance the pushability and trackability of the catheter shaft 400 independently of the stiffening structure associated with the side aperture 451. This optional use of an extra stiffening member such as the wire guide 480 to provide enhanced stiffness proximally of the side aperture 451 may be used with the other embodiments illustrated herein as well as with other embodiments within the scope of the present invention.

In the embodiments illustrated in FIGS. 4A-4E as well as alternative embodiments, the stiffening structure (406, 410, 412, 450, and 456) is preferably stiffer than the material comprising the catheter shaft 400 in the region of the side aperture 408. The stiffening structure therefore provides a stiffening effect for the catheter shaft 400, thereby biasing it against undesired or excessive flexure. For example, the stiffening structure (406, 410, 412, 450, 456) may comprise a metal, plastic, or other material that is stiffer (e.g., more rigid) than the plastic or other material that comprises the wall of the catheter shaft 400. The stiffening structure may also comprise a structural shape that will confer enhanced stiffness to the catheter shaft 400, e.g., by increasing the thickness of the shaft wall at the location most likely to exhibit kinking or excessive bending. In other alternative embodiments, the stiffening structures described in FIGS. 4A-4E may be disposed within the wall of the catheter shaft 400 as opposed to merely lining the interior surface of the shaft wall.

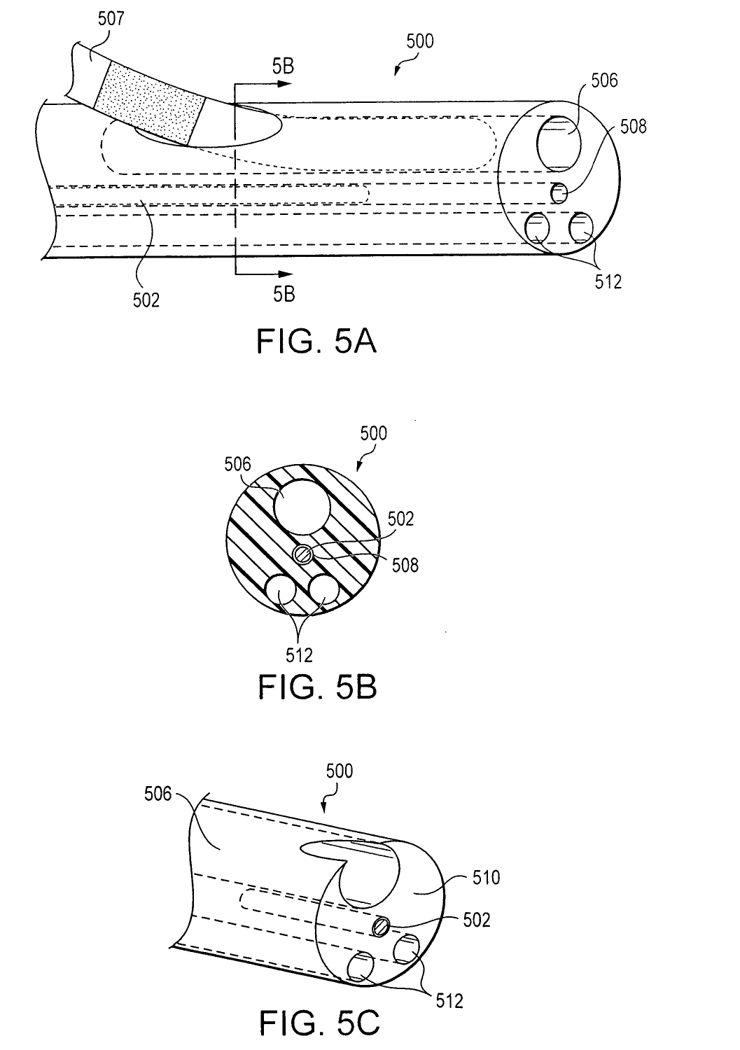

FIG. 5A illustrates a perspective view of an embodiment of a multi-lumen catheter shaft 500 having a stiffening structure in the form of a stylet comprising a wire 502 made of, for example, nitinol or another suitable stiffening material disposed in a central lumen of a multi-lumen catheter. The wire 502 traverses the area immediately adjacent the side port aperture 504 (which opens to a wire guide lumen 506). A wire guide 507 is shown being advanced through the wire guide lumen 506. FIG. 5B illustrates a transverse cross-sectional view of the catheter along line 5B-5B of FIG. 5A showing multiple lumens in the catheter shaft 500, including a central lumen 508. As shown in FIG. 5A, the wire 502 may extend substantially proximally of the side port aperture 504, or, as shown in FIG. 5C, the wire 502 may extend only slightly proximally of the side port aperture 504. In the embodiment illustrated, wire 502 is disposed within a central lumen 508. Alternatively, and as shown in FIG. 5C, the wire 502 may be disposed in a substantially solid central portion of the catheter shaft 500 that separates and forms a septum 510 between wire guide lumen 506 and secondary lumens 512. As another alternative, the wire 502 may be disposed at least partially in the wall of a single- or multi-lumen catheter.

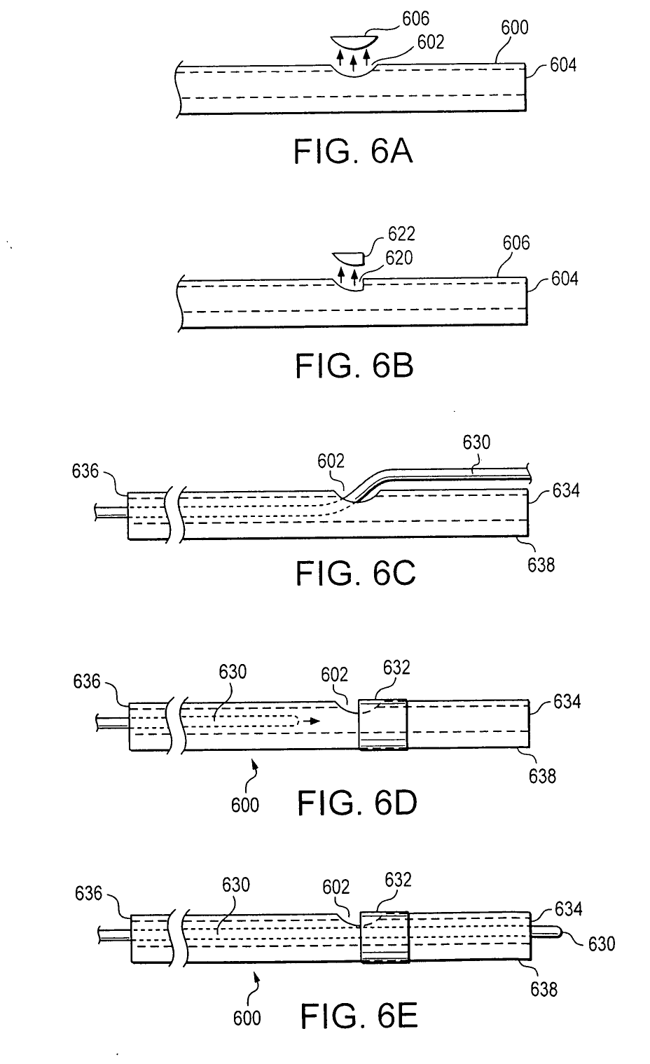

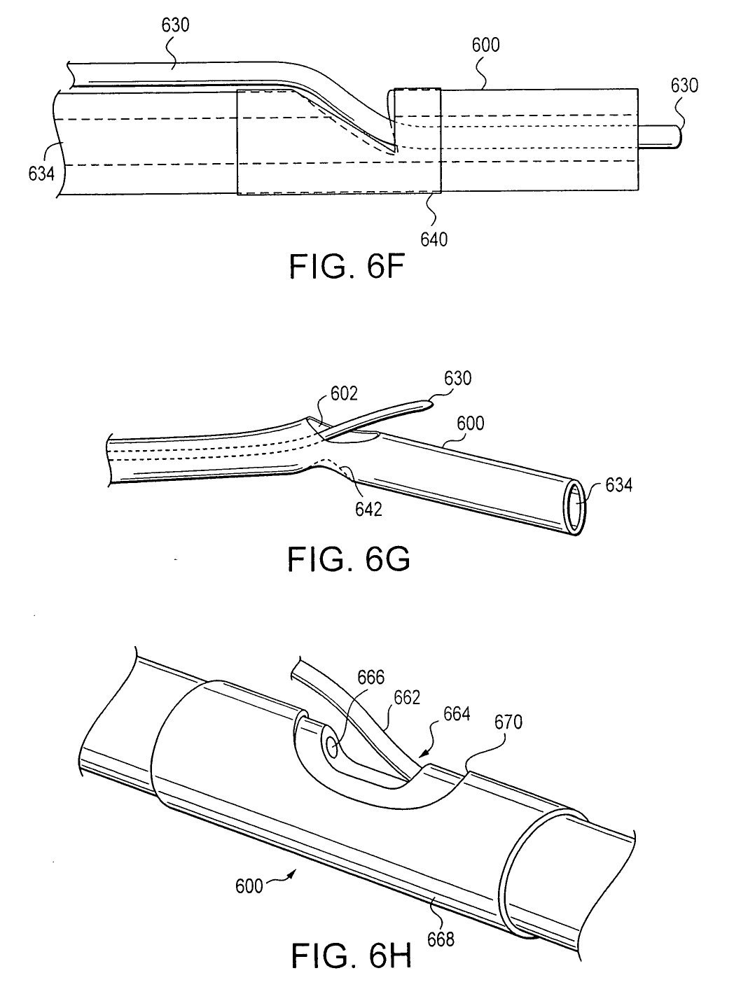

FIGS. 6A-6F illustrate a device and method for making a side port aperture and providing support around the side port aperture in a convertible/dual use catheter shaft. As with all of the stiffening-enhancing embodiments described herein, the device embodiments shown in FIGS. 6A-6F may be used in short wire, long wire, or convertible/dual use catheter configurations. FIG. 6A illustrates one method of creating a side port aperture 602 open to a wire guide lumen 604 in a catheter shaft 600 by skiving out an oval section 606. This skiving may be accomplished, for example, with a cutting blade or with a rotating drill bit applied transversely across the surface of the catheter shaft 600. The skived-out section 606 can be a shape other than oval, including asymmetric shapes, that will provide a suitably shaped side port aperture 602. For example, FIG. 6B shows an alternative method of making a side port aperture 620 having a wedge-like shape of a special ungula of a substantially right circular cylinder (where the body of the catheter shaft 600 is the substantially right circular cylinder). More specifically, a side port aperture having this shape requires that the portion cut away 622 from the catheter shaft 600 (to form the side port aperture 620) have a generally parabolic shape 622. Alternatively, the side port aperture 620 may have a different wedge-like shape or some other appropriate shape. It should be appreciated that the manufacture of such alternative side port aperture shapes will require the use of a specially shaped blade, multiple cuts, or both.

FIGS. 8A-8D illustrate methods of making alternative side port aperture shapes. FIG. 8A illustrates a catheter shaft 800 having two lumens 802, 804. The lumen 802 is separated from the exterior of the shaft 800 by a wall 801. In the illustrated embodiment of a method of making a side port aperture, a rotating drill bit 806 is reciprocatingly guided several times through an upper surface of the shaft 800 and into the lumen 802, creating a side port aperture 808 consisting of overlapping holes formed by the drill bit 806. As shown in the partially rotated perspective view of FIG. 8B, the side port aperture 808 is open to the lumen 802. The region of the catheter 800 near the aperture 808 may then be reinforced using one or more of the stiffening structures and methods described herein. The opening of the aperture has a lip that includes a cross-sectional surface 810 of the shaft wall 801. The drilling method creates multiple faces 812 in the surface 810. In this embodiment, all faces 812 of the aperture 808 are parallel to each other and perpendicular to a plane 814 that intersects the longitudinal axis of the lumen 802. In an alternative embodiment of this method, only a single drill puncture is made, creating a single round aperture.

FIGS. 8C-8D illustrate a similar method of making a side port aperture. As shown in FIG. 8C, a router bit 824 is directed into the upper surface of a catheter shaft 820 and moved along the longitudinal axis of the shaft 820 for a pre-selected distance. The side perspective of FIG. 8D shows that the resulting side port aperture 828 is open to a central lumen 822 of the shaft 820. Those of skill in the art will appreciate that other embodiments of the methods illustrated in FIGS. 8A-8D are possible and are within the scope of the present invention. For example, a gouging cutting tool can be used rather than a rotating blade cutter.

FIG. 6C illustrates the dual use catheter shaft 600 with a wire guide 630 being advanced distally therethrough (i.e., toward the distal end 638 of the shaft 600) and erroneously passing through the side port aperture 602. It is one goal of the invention described herein to prevent such erroneous wire guide placement from occurring by promoting a desired directional passage of the wire guide 630 along a desired route toward the distal end of the catheter shaft 600. As will be explained in greater detail below, the possibility of erroneous wire guide placement can be reduced by modifying the shape of side port aperture 602. FIG. 6B illustrates an example of a side port aperture 620 that reduces the possibility of erroneous wire guide placement. Other examples are described below.

FIGS. 6D-6F illustrate a dual use catheter shaft 600 with a band 632 placed thereupon to partially cover the distal portion of side port aperture 602. As shown in FIGS. 6D-6E, when a wire guide 630 is being advanced through the wire guide lumen 634 from the proximal 636 toward the distal 638 end in the long wire application of the dual use catheter shaft 600, the placement of the band 632 reduces the likelihood that the wire guide 630 will exit through side port aperture 602 (as shown in FIG. 6C). This reduction of improper tracking of the wire guide 630 is aided in at least one of a couple ways: (1) the shape of the aperture 602 combined with the presence of the support band 632 at least partially occludes the side port aperture 602 to prevent undesired passage of the wire guide 630; and (2) the stiffening effect of the support band 632 (or other stiffening structure) on the catheter 600 reduces the likelihood of undesired flexure of the catheter shaft 600 in the region of the side port aperture 602 that would promote mistracking of the wire guide 632. For example, FIG. 6G illustrates a catheter shaft 600 without any stiffening structure and exhibiting undesired flexure 642 in the region of the side port aperture 602, causing mistracking of the wire guide 632 such that it improperly exits the side port aperture 632 instead of proceeding distally to the end of the catheter 600 in an appropriate fashion.

FIG. 6F illustrates how a wire guide 630 may still be directed through the side port aperture 602 for use of the dual use catheter shaft 600 in a “short wire” configuration. The support band 640 in FIG. 6F is one of several different possible shapes that may be used for partially occluding the side port aperture 642 to promote proper tracking of the wire guide 630 while enhancing the stiffness of the catheter shaft 600 in the region of the side port aperture 602.

FIG. 6H illustrates another embodiment of a catheter shaft 660 with a wire guide 662 directed through a side port aperture 664 into a wire guide lumen 666 of the catheter shaft 660. The region of the side port aperture 664 is reinforced by a support band 668 that has an opening 670 corresponding to the side port aperture 664, and that substantially surrounds the circumference of the catheter 660.

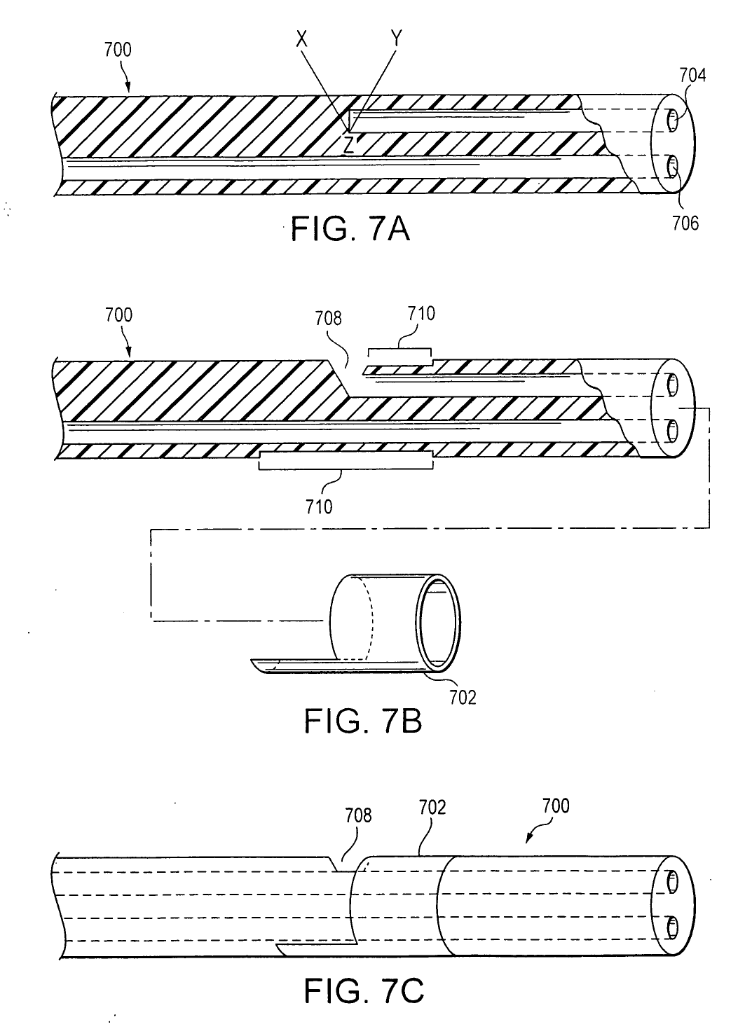

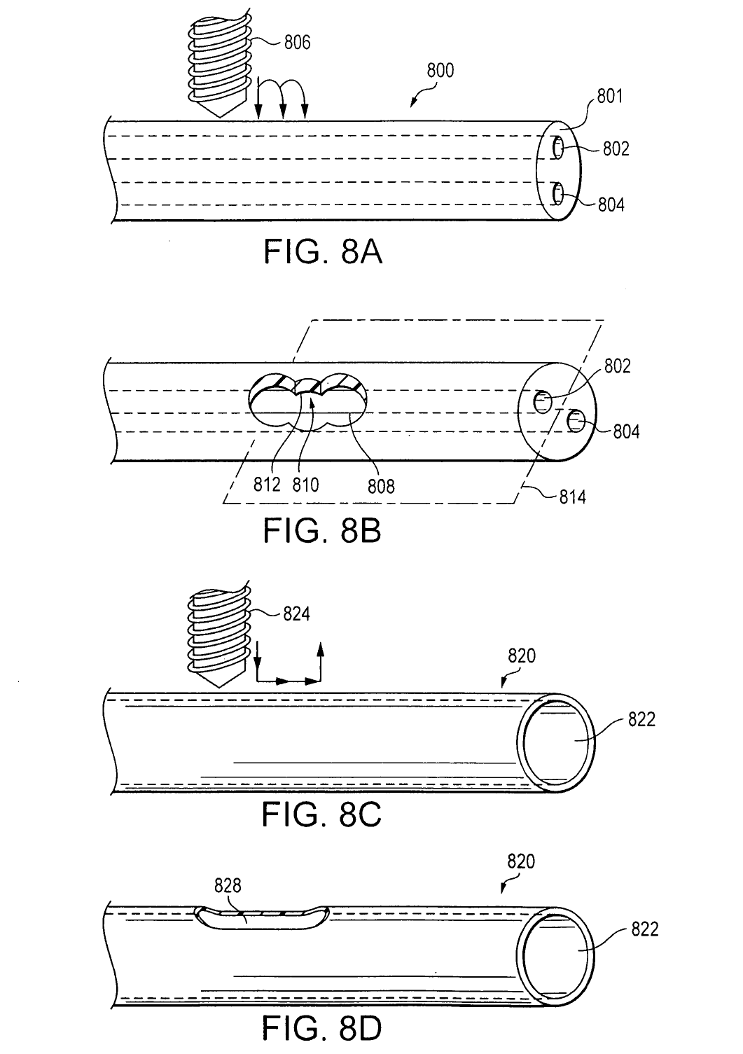

FIGS. 7A-7C illustrate a method for placing a support band 702 onto a catheter shaft 700. FIGS. 7A and 7B each show a longitudinal cross sectional view of the distal end of a catheter shaft 700. The shaft 700 has a short wire guide lumen 704 and a primary lumen 706 that extends toward the proximal end of the catheter shaft 700. As indicated in FIG. 7A, separate cuts are made along lines X-Z and Y-Z to form a side port aperture 708 opening into wire guide lumen 704. As shown in FIG. 7B, a thin portion of the exterior wall of catheter shaft 700 is removed so as to create a surface indentation 710. The shape and size of the surface indentation 710 corresponds to the shape and size of the support band 702. The support band 702 is mounted by sliding it over the end of the catheter shaft 700. This mounting step can occur over either end of the catheter shaft 700. The support band 702 is aligned with the indentation 710 and crimped into place. Because of the shape and position of the support band 702 relative to the indentation 710, the crimped-in-place support band 702 does not significantly increase the outer diameter of the overall assembly with catheter shaft 700. FIG. 7C illustrates the support band 702 assembled to the catheter shaft 700. In alternatives to the method described above, one may forgo making the indentation 710. The support band 702 may likewise comprise a different shape than illustrated. For example, the support band 702 may initially be an open, flat band that is molded or crimped into position around the catheter shaft 700 adjacent the side port aperture 708. The band 702 may be mounted onto the catheter shaft 700 with adhesive or by some other method as well.

Many of the different embodiments of support structures described above may be varied further or used in combination with each other. For example, a catheter shaft 600 having a support band 632 as illustrated in FIGS. 6D-6E may also include a stiffening composition (as described in connection with FIG. 2A) disposed on an interior or exterior surface of the catheter shaft 600, or on a lip of the side port aperture 603. As another example, a stiffening composition may be applied around a side port aperture (as in FIG. 2B) in a catheter shaft having tubular or cannular stiffening structure (as in FIGS. 4D-4E). As yet another illustration of alternative embodiments of materials, various of the stiffening structures can be constructed from, for example, NiTi, nitinol, deformable plastic, aluminum, a fiber-reinforced composite, a particulate-reinforced composite, or stainless steel. Other combinations and variations of the embodiments disclosed herein will be readily apparent to those skilled in the art.

The materials and methods appropriate for use with the foregoing embodiments of the present invention but not explained in detail herein will be readily apparent to those skilled in the art. It is therefore intended that the foregoing detailed description be regarded as illustrative rather than limiting, and that it be understood that it is the following claims, including all equivalents, that are intended to define the spirit and scope of this invention.

We claim:

1. A catheter, comprising an elongate shaft, said elongate shaft comprising:
a proximal end;
a distal end;
a first lumen extending through at least a portion of the shaft and defined by a wall;
an aperture through the wall, open to the first lumen;
said aperture disposed between the proximal and distal ends;
an outer circumference; and
stiffening structure disposed near the aperture for preventing undesired flexure.

2. The catheter of claim 1, wherein the stiffening structure comprises:
a composition disposed in a placement selected from on an interior surface of the first lumen rimming the aperture, on the interior surface immediately adjacent the aperture, on the interior surface opposite the aperture, on an exterior surface of the wall surrounding the aperture, on an exterior surface immediately adjacent the aperture, on an exterior surface opposite the aperture, and any combination thereof,
said composition providing a stiffening of the surface on which the composition is disposed, said stiffening of the surface enhancing a columnar strength stiffness of the catheter shaft near the aperture.

3. The catheter of claim 2, wherein the composition is selected from a group consisting of a polymer, a composite, a suspension, a solution, a solvent, and a material substantially the same as a material comprising the shaft.

4. The catheter of claim 2, wherein the composition comprises a particulate material.

5. The catheter of claim 1, wherein the stiffening structure comprises at least one stiffening stylet, with at least one of a proximal and distal end near the aperture.

6. The catheter of claim 5, wherein the at least one stiffening stylet is at least partially disposed within the shaft wall or within the first lumen.

7. The catheter of claim 5, further comprising a second lumen, wherein the at least one stiffening stylet is disposed within the second lumen.

8. The catheter of claim 5, wherein the at least one stiffening stylet comprises material selected from the group consisting of NiTi, nitinol, deformable plastic, aluminum, a fiber-reinforced composite, a particulate-reinforced composite and stainless steel.

9. The catheter of claim 5, wherein the at least one stiffening stylet is disposed on the exterior surface.

10. The catheter of claim 9, wherein the at least one stiffening stylet is secured to the exterior surface by adhesive or by at least one band around the stylet and at least a portion of the outer circumference of the shaft.

11. The catheter of claim 1, wherein the stiffening structure comprises at least one stiffening tab or at least one support band.

12. The catheter of claim 11, comprising the at least one support band, wherein the support band surrounds the outer circumference and is disposed about the exterior surface from a point proximal of the aperture to a point distal of the aperture, and an opening in the support band corresponds to the aperture.

13. The catheter of claim 11, comprising the at least one support band, wherein the at least one support band comprises a first band proximal of the aperture substantially surrounding the outer circumference, a second band distal of the aperture substantially surrounding the outer circumference, with at least one support member disposed therebetween.

14. The catheter of claim 11, comprising the at least one support band, wherein the at least one support band comprises a first band proximal of the aperture disposed in and substantially surrounding a radial portion of a first interior surface of the lumen, a second band distal of the aperture disposed in and substantially surrounding a radial portion of the first interior surface, with at least one support member disposed therebetween.

15. The catheter of claim 11, comprising the at least one support band, wherein the at least one support band comprises a first band proximal of the aperture disposed in the wall and substantially surrounding a radial portion of a first interior surface of the lumen, a second band distal of the aperture disposed in the wall and substantially surrounding a radial portion of the first interior surface of the lumen, with at least one support member disposed therebetween.

16. The catheter of claim 11, comprising the at least one support band, wherein the at least one support band is a semi-cylindrical support band.

17. The catheter of claim 16, comprising the at least one support band, wherein the semi-cylindrical support band is disposed on the exterior surface, within the first lumen, or within the wall

18. The catheter of claim 1, wherein the stiffening structure comprises at least one cannula.

19. The catheter of claim 18, comprising the at least one cannula being disposed about a first interior surface of the lumen from a point proximal of the aperture to a point distal of the aperture, with an opening in the cannula corresponding to the aperture.

20. The catheter of claim 28, comprising the at least one cannula being disposed within the wall from a point proximal of the aperture to a point distal of the aperture, with an opening in the cannula corresponding to the aperture.

21. The catheter of claim 1, further comprising at least one second lumen separated from the first lumen by a septum.

22. The catheter of claim 21, wherein the stiffening structure is disposed in the second lumen or in the septum.

23. The catheter of claim 21, wherein the stiffening structure is disposed on an inner surface of the second lumen.

24. The catheter of claim 21, wherein the stiffening structure is integrated at least partially within the wall around the second lumen.

25. The catheter of claim 1, wherein the stiffening structure comprises a portion of the catheter shaft that has been exposed to and altered by an energy, a chemical agent, or a combination thereof.

26. The catheter of claim 25, wherein the energy is selected from a group consisting of heat, visible light, infrared light, ultraviolet light, RF energy, microwaves, X-rays, ultrasound waves, and any combination thereof.

27. A method of forming a reinforced aperture in a shaft of a catheter for promoting a desired directional passage of a wire guide comprising the steps of:
(A) providing a catheter having a shaft comprising:
a first lumen defining an interior surface,
a proximal end,
a distal end,
an outer circumference, and
an exterior surface;
(B) cutting the exterior surface near the distal end to form an aperture open from the exterior surface to the first lumen; and
(C) providing a reinforcing band immediately adjacent the aperture.

28. The method of claim 27, wherein a proximal portion of the band provided in step (C) is position covers at least a distal portion of the aperture such that an uncovered portion of the aperture forms a special ungula of the substantially right circular cylinder having a distal arc substantially normal the longitudinal axis and an intersecting proximal arc at an angle oblique to the longitudinal axis.

29. A method for creating an aperture in a catheter shaft comprising the steps of:
providing a catheter shaft having at least one lumen defined by a wall of the shaft;
providing a cutting bit having a cutting diameter that is not greater than an outside diameter of the catheter shaft; and
directing a portion of the bit through the wall to form an aperture open into the lumen.

30. The method of claim 29, wherein the cutting bit is a rotatable cutting bit and the directing step includes rotating the cutting bit.

31. The method of claim 29, wherein the cutting bit is a gouging tool.
